(19) United States
(12) Patent Application Publication
(10) Pub. No.: US 2022/0288386 A1
(43) Pub. Date: Sep. 15, 2022

(54) CURRENT STIMULATION DEVICE AND OSTEOBLAST DIFFERENTIATION TREATMENT SYSTEM THROUGH CURRENT STIMULATION

(71) Applicant: The Second Affiliated Hospital of Soochow University, Suzhou (CN)

(72) Inventors: Xiaozhong ZHOU, SUZHOU (CN); Yingzi ZHANG, SUZHOU (CN); Zhou LI, SUZHOU (CN); Lingling XU, SUZHOU (CN); Zhuo LIU, SUZHOU (CN)

(21) Appl. No.: 17/514,169

(22) Filed: Oct. 29, 2021

(30) Foreign Application Priority Data
Mar. 10, 2021 (CN) 202110260205.3

(51) Int. Cl. A61N 1/32; A61N 1/05; H02N 1/04

(52) U.S. Cl. CPC A61N 1/326; A61N 1/0502; H02N 1/04

(57) ABSTRACT

A current stimulation device and an osteoblast differentiation treatment system through current stimulation are provided, which relate to the technical field of bioengineering. The current stimulation device includes a nanogenerator, which is electrically connected to a stimulation electrode. The stimulation electrode makes contact with an affected part. The stimulation of the present disclosure is applied to the affected part of a fracture patient, and realizes the current stimulation through the nanogenerator, promotes proliferation and activity of the osteoblast. The present disclosure provides a new idea for fracture healing, and promotes application progress of the nanogenerator in a wearable electronic medical instrument. The present disclosure has portability and an excellent clinical application prospect.

### CROSS REFERENCE TO RELATED APPLICATION

This patent application claims the benefit and priority of Chinese Patent Application No. 202110260205.3 filed on Mar. 10, 2021, the disclosure of which is incorporated by reference herein in its entirety as part of the present application.

### TECHNICAL FIELD

The present disclosure relates to the technical field of bioengineering, and in particular to a current stimulation device and an osteoblast differentiation treatment system through current stimulation.

### BACKGROUND ART

Bone has the function of movement, support and body protection, which is of great significance to maintain the normal activities of the human body. Fracture is a common clinical disease that leads to complete or partial breakage of bone structural continuity, and mainly occurs to children and the elderly. The healing process of fractures will last for a long time, and is susceptible to the interference of many factors, resulting in delayed union or even nonunion, which seriously affects the quality of life of patients. Therefore, how to promote fracture repair and shorten healing time is the forefront and difficulty of orthopedic research.

With the in-depth study of the fracture healing mechanism, various adjuvant treatment methods have been widely applied in clinical practice. For example, bone tissue engineering (BTE) simulates autogenous bone graft in many aspects, uses stents and osteoblasts to fill the defective bone, and adds growth factors or electrical stimulation to regulate the cell-cell interaction and cell-stent interaction. In particular to the physical therapy of electrical stimulation, its function in promoting fracture healing has been fully confirmed. Electrical stimulation can not only promote the healing of fresh fracture, but also has excellent curative effect on delayed union of fracture, bone nonunion, osteotomy, pseudarthrosis formation.

It is a key factor in the process of bone remodeling and repair to promote osteogenesis through electrical stimulation. Electrical stimulation can be divided into current stimulation, electrical field stimulation and electromagnetic field stimulation. However, electrical stimulation devices are too bulky for clinical treatment. It is still an enormous challenge to develop a portable and highly patient-compliant electrical stimulation treatment device for bone repair.

### SUMMARY

The purpose of the present disclosure is to provide a current stimulation device and an osteoblast differentiation treatment system through current stimulation, so as to solve the above problem existing in the prior art, which are conducive to a bone repair process and have excellent flexibility and portability.

In order to achieve the above-mentioned purpose, the present disclosure provides the following solutions.

The present disclosure provides a current stimulation device. The current stimulation device includes a nanogenerator. The nanogenerator is electrically connected to a stimulation electrode, and the stimulation electrode makes contact with an affected part.

In some embodiments, the nanogenerator may be shaped as an arch.

In some embodiments, a rectifier bridge may be arranged between the nanogenerator and the stimulation electrode.

In some embodiments, the nanogenerator may be a piezoelectric nanogenerator and include a Kapton film. The Kapton film may be formed by thermoforming, a lower surface of the Kapton film may be sequentially coated with a first silver electrode and a second silver electrode, and the first silver electrode and the second silver electrode each may be electrically connected to the stimulation electrode by means of a wire.

In some embodiments, an upper surface of a polyvinylidene fluoride (PVDF) film may be coated with the first silver electrode, a lower surface of the PVDF film may be coated with the second silver electrode, and a lower surface of the second silver electrode may be coated with a polyethylene terephthalate (PET) film.

In some embodiments, the Kapton film, the first silver electrode, the PVDF film, the second silver electrode and the PET film may be sequentially bonded by means of a silicone polymer.

In some embodiments, the nanogenerator may be a triboelectric nanogenerator.

In some embodiments, the stimulation electrode may be a needle electrode.

In some embodiments, the stimulation electrode may include a positive electrode and a negative electrode. The negative electrode may be connected to a wound broken end of the affected part, and the positive electrode may be connected to muscle tissue adjacent to the wound broken end.

The present disclosure further provides an osteoblast differentiation treatment system through current stimulation. The system may include the current stimulation device. Connecting structures may be arranged at left and right ends of the nanogenerator, and may be connected to a splint.

Compared with the prior art, the present disclosure achieves the following technical effects.

The stimulation of the present disclosure is applied to the affected part of a fracture patient, and realizes the current stimulation through the nanogenerator, promotes proliferation and activity of the osteoblast. The present disclosure provides a new idea for fracture healing, and promotes application progress of the nanogenerator in a wearable electronic medical instrument. The present disclosure has portability and an excellent clinical application prospect.

### BRIEF DESCRIPTION OF THE DRAWINGS

In order to more clearly illustrate the embodiments of the present disclosure or the technical solutions in the prior art, the accompanying drawings used in the embodiment will be briefly described below. Apparently, the accompanying drawings in the following description are only some embodiments of the present disclosure, and those ordinary skilled in the art can obtain other drawings according to the drawings without creative efforts.

FIG. 1 is a schematic diagram of a current stimulation device according to the present disclosure;

FIG. 2 is a schematic diagram of a nanogenerator according to the present disclosure;

FIG. 3 is a schematic structural diagram of each layer of the nanogenerator according to the present disclosure; and

FIG. 4 is a schematic diagram of an application of an osteoblast differentiation treatment system through current stimulation according to the present disclosure.

List of reference characters: 100 current stimulation device; 200 osteoblast differentiation treatment system through current stimulation; 1 nanogenerator; 2 rectifier bridge; 3 stimulation electrode; 4 Kapton film; 5 first silver electrode; 6 second silver electrode; 7 wire; 8 PVDF film; 9 PET film; and 10 connecting structure.

### DETAILED DESCRIPTION OF THE EMBODIMENTS

The technical solutions in the embodiments of the present disclosure will be clearly and completely described with reference to the drawings in the embodiments of the present disclosure. Apparently, the described embodiments are only a part of the embodiments of the present disclosure, and not all of the embodiments. On the basis of the embodiments of the present invention, all other embodiments obtained by those of ordinary skills in the art without inventive efforts all fall within the scope of protection of the present disclosure.

The embodiments aim to provide a current stimulation device and an osteoblast differentiation treatment system through current stimulation, so as to solve the above problem existing in the prior art, which are conducive to a bone repair process and have excellent flexibility and portability.

To make the above-mentioned objectives, features, and advantages of the present disclosure more comprehensible, the present disclosure will be further described in detail below with reference to the drawings and particular embodiments.

### Embodiment 1

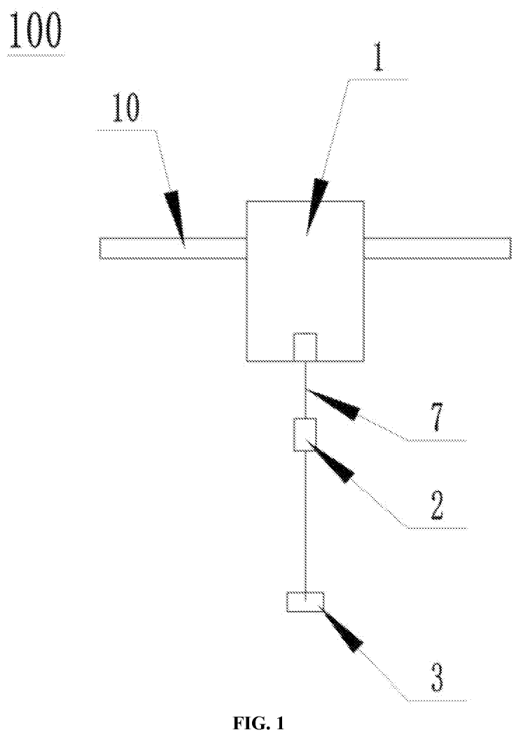

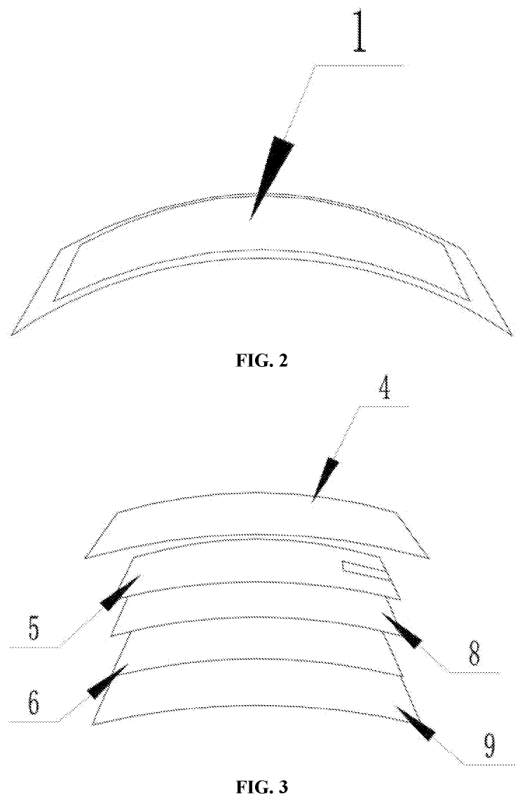

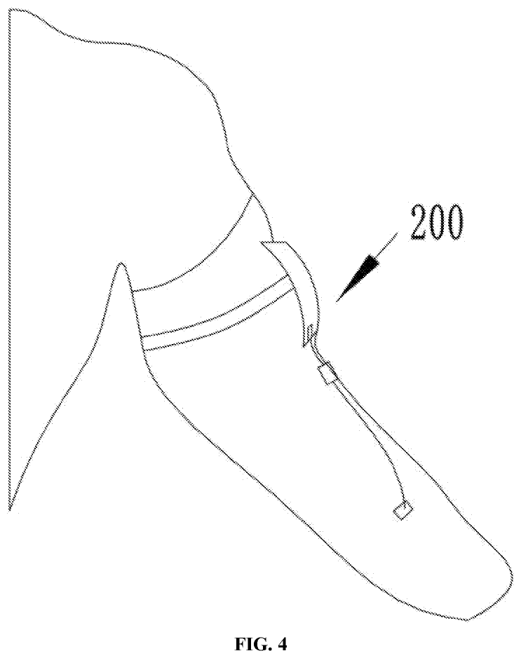

As shown in FIGS. 1 to 3, this embodiment provides a current stimulation device 100. The current stimulation device includes a nanogenerator 1 (shape-memory piezoelectric nanogenerator (sm-PENG)) which can convert biomechanical energy into electrical energy. The nanogenerator 1 is electrically connected to a stimulation electrode 3, and the stimulation electrode 3 makes contact with an affected part. A current range of electrical stimulation output by the nanogenerator 1 may be nA to mA level, and a frequency range may be from one Hz to thousands of Hz. The stimulation of this embodiment is applied to the affected part of a fracture patient, and realizes the current stimulation through the nanogenerator 1, promotes proliferation and activity of the osteoblast. This embodiment provides a new idea for fracture healing, and promotes application progress of the nanogenerator 1 in a wearable electronic medical instrument. This embodiment has portability and an excellent clinical application prospect.

Specifically, in this embodiment, the nanogenerator 1 is shaped as an arch. The arch structure greatly improves electrical output performance of the nanogenerator 1, short-circuit current may reach 20 μA by tapping the nanogenerator 1, which is more than twice that of a flat plate structure. The output current may better meet requirements of electrical stimulation treatment.

In some embodiments, a rectifier bridge 2 is arranged between the nanogenerator 1 and the stimulation electrode 3. An alternating current pulse electrical signal is output by the nanogenerator 1 under a condition of not passing through the rectifier bridge 2; and a direct current pulse electrical signal is output by the nanogenerator 1 under a condition of passing through the rectifier bridge 2.

In some embodiments, the nanogenerator 1 is a piezoelectric nanogenerator. The nanogenerator 1 includes a Kapton film 4. The Kapton film 4 is formed by thermoforming, so as to form the arched Kapton film 4. A lower surface of the Kapton film 4 is sequentially coated with a first silver electrode 5 and a second silver electrode 6. The first silver electrode 5 and the second silver electrode 6 each are electrically connected to the rectifier bridge 2 by means of a wire 7.

In some embodiments, an upper surface of a polyvinylidene fluoride (PVDF) film 8 is coated with the first silver electrode 5, a lower surface of the PVDF film 8 is coated with the second silver electrode 6, and a lower surface of the second silver electrode 6 is coated with a polyethylene terephthalate (PET) film 9.

In some embodiments, the Kapton film 4, the first silver electrode 5, the PVDF film 8, the second silver electrode 6 and the PET film 9 are sequentially bonded by means of a silicone polymer.

In some embodiments, a manufacturing process of the nanogenerator 1 is as follows:

Firstly, a heating rod is used for thermally forming the Kapton film 4 with a dimension of 55×25×0.1 mm³ into an arch at about 200° C.; after formation, the upper and lower surfaces of PVDF film 8 are respectively coated with the first silver electrode 5 and the second silver electrode 6 (the dimensions are both 50×20×0.11 mm³), and then the PVDF film is attached to the arched Kapton film 4. Then, the PET film 9 is used as a packaging layer for packaging; and finally, interlayer structures are bonded together one by one by means of the silicone polymer, to manufacture the nanogenerator 1 (sm-PENG).

In some embodiments, the stimulation electrode 3 is a needle electrode. The stimulation electrode 3 includes a positive electrode and a negative electrode. The negative electrode is connected to a wound broken end (fracture gap) of the affected part, and the positive electrode is connected to muscle tissue adjacent to the wound broken end.

The current stimulation device 100 of this embodiment stimulates osteoblast differentiation by means of self-powered pulsed direct current. The pulsed direct current (DC) of the nanogenerator 1 promotes osteogenesis of mouse embryo osteoblast precursor cells (MC3T3-E1) and may inhibit osteoclasts.

The current stimulation device 100 of this embodiment may effectively promote proliferation of osteoblasts, promote the activity of calcium ions in osteoblasts by means of the pulsed DC generated by the rectifier bridge 2, and have a certain osteoblasts orientation effect. Moreover, the current stimulation device may promote activity of alkaline phosphatase (ALP) of the osteoblasts under a condition of long-term culture, and finally promotes calcium deposition, extracellular matrix mineralization and osteoblast differentiation. A biological effect of the current stimulation generated by the pulsed direct current of the nanogenerator 1 is basically the same as that of a commercial signal generator. This embodiment provides a new idea for fracture healing, and promotes application progress of the nanogenerator 1 in a wearable electronic medical instrument.

### Embodiment 2

A difference between this Embodiment and Embodiment 1 lies in that the nanogenerator 1 is a triboelectric nanogenerator.

### Embodiment 3

As shown in FIG. 4, this embodiment provides an osteoblast differentiation treatment system through current stimulation 200. The system includes the current stimulation device 100 of Embodiment 1 or 2. Connecting structures 10 are arranged at left and right ends of the nanogenerator 1. The connecting structures 10 are bandages, and connected to a splint. The connecting structures 10 may be connected to gauzes, then the gauzes are connected to the splint. The osteoblast differentiation treatment system through current stimulation 200 is formed by the combination of the current stimulation device 100 and the splint, which has excellent flexibility and portability.

Several examples are used in this specification for illustration of the principles and implementation methods of the present disclosure. The description of the above embodiments is merely used to help understand the method and its core concept of the present disclosure. In addition, those skilled in the art may make modifications to the specific embodiments and scope of application in accordance with the teachings of the present disclosure. In conclusion, the content of this specification should not be construed as a limitation to the present disclosure.

What is claimed is:

1. A current stimulation device, comprising a nanogenerator, wherein the nanogenerator is electrically connected to a stimulation electrode, and the stimulation electrode makes contact with an affected part.

2. The current stimulation device according to claim 1, wherein the nanogenerator is shaped as an arch.

3. The current stimulation device according to claim 1, wherein a rectifier bridge is arranged between the nanogenerator and the stimulation electrode.

4. The current stimulation device according to claim 1, wherein the nanogenerator is a piezoelectric nanogenerator and comprises a Kapton film, the Kapton film is formed by thermoforming, a lower surface of the Kapton film is sequentially coated with a first silver electrode and a second silver electrode, and the first silver electrode and the second silver electrode each are electrically connected to the stimulation electrode by means of a wire.

5. The current stimulation device according to claim 4, wherein an upper surface of a polyvinylidene fluoride (PVDF) film is coated with the first silver electrode, a lower surface of the PVDF film is coated with the second silver electrode, and a lower surface of the second silver electrode is coated with a polyethylene terephthalate (PET) film.

6. The current stimulation device according to claim 5, wherein the Kapton film, the first silver electrode, the PVDF film, the second silver electrode and the PET film are sequentially bonded by means of a silicone polymer.

7. The current stimulation device according to claim 1, wherein the nanogenerator is a triboelectric nanogenerator.

8. The current stimulation device according to claim 1, wherein the stimulation electrode is a needle electrode.

9. The current stimulation device according to claim 1, wherein the stimulation electrode comprises a positive electrode and a negative electrode, the negative electrode is connected to a wound broken end of the affected part, and the positive electrode is connected to muscle tissue adjacent to the wound broken end.

10. An osteoblast differentiation treatment system through current stimulation, comprising a current stimulation device,
the current stimulation device comprising a nanogenerator, wherein the nanogenerator is electrically connected to a stimulation electrode, and the stimulation electrode makes contact with an affected part;
connecting structures are arranged at left and right ends of the nanogenerator, and connected to a splint.

11. The treatment system according to claim 10, wherein the nanogenerator is shaped as an arch.

12. The treatment system according to claim 10, wherein a rectifier bridge is arranged between the nanogenerator and the stimulation electrode.

13. The treatment system according to claim 10, wherein the nanogenerator is a piezoelectric nanogenerator and comprises a Kapton film, the Kapton film is formed by thermoforming, a lower surface of the Kapton film is sequentially coated with a first silver electrode and a second silver electrode, and the first silver electrode and the second silver electrode each are electrically connected to the stimulation electrode by means of a wire.

14. The treatment system according to claim 13, wherein an upper surface of a polyvinylidene fluoride (PVDF) film is coated with the first silver electrode, a lower surface of the PVDF film is coated with the second silver electrode, and a lower surface of the second silver electrode is coated with a polyethylene terephthalate (PET) film.

15. The treatment system according to claim 14, wherein the Kapton film, the first silver electrode, the PVDF film, the second silver electrode and the PET film are sequentially bonded by means of a silicone polymer.

16. The treatment system according to claim 10, wherein the nanogenerator is a triboelectric nanogenerator.

17. The treatment system according to claim 10, wherein the stimulation electrode is a needle electrode.

18. The treatment system according to claim 10, wherein the stimulation electrode comprises a positive electrode and a negative electrode, the negative electrode is connected to a wound broken end of the affected part, and the positive electrode is connected to muscle tissue adjacent to the wound broken end.
